(19) United States
(12) Patent Application Publication
(10) Pub. No.: US 2008/0146861 A1
(43) Pub. Date: Jun. 19, 2008

(54) Brachytherapy Shielding System

(75) Inventors: Brent D. Murphy, South Bend, IN (US); James Wheeler, Goshen, IN (US)

Correspondence Address: BARNES & THORNBURG LLP, 11 SOUTH MERIDIAN, INDIANAPOLIS, IN, 46204, US

(73) Assignee: GOSHEN HEALTH SYSTEM, INC., Goshen, IN (US)

(21) Appl. No.: 11/877,424

(22) Filed: Oct. 23, 2007

Related U.S. Application Data
(60) Provisional application No. 60/875,464, filed on Dec. 18, 2006.

(51) Int. Cl. A61N 5/00; A61B 5/05

(52) U.S. Cl. 600/3; 600/424

(57) ABSTRACT

A brachytherapy applicator shielding system includes a brachytherapy applicator, a magnet, and magnetically attractable particles. The brachytherapy applicator shielding system is arranged to shield skin tissue exposed to a radiation source during intracavitary radiation therapy.

This application claims priority under U.S.C. 517 119(e) to U.S. Provisional Application Ser. No. 60/875,464, filed Dec. 18, 2006, which is expressly incorporated by reference herein.

### BACKGROUND

The present disclosure relates to an applicator and particularly to a brachytherapy applicator. More particularly, the present disclosure is related to a brachytherapy applicator for intracavitary radiation therapy.

In some instances, the ability to perform intracavitary brachytherapy is contingent upon the patient's “skin thickness” adjacent to a lumpectomy cavity. Care providers may be reluctant to provide this procedure when a skin distance (the distance between an inserted brachytherapy applicator and an external skin surface) is less than 7 mm. For example, a small skin distance, such as a skin distance less than 5 mm, may increase the risk of radiation induced burns to the skin tissue adjacent to the radiation source. Illustratively, therefore it may be desirable for the skin distance to be at least 10 mm or more.

### SUMMARY

According to the present disclosure, a brachytherapy shielding system includes a brachytherapy applicator. The brachytherapy applicator is configured to provide localized radiation therapy to remaining tissue after a cancerous lump or growth has been removed from the body of a patient.

In illustrative embodiments, the brachytherapy applicator shielding system also includes magnetically attractable shielding particles, and a magnet. The brachytherapy applicator is implanted into the surgical cavity in the patient. The magnetically attractable shielding particles are emplaced in the brachytherapy applicator and attracted to the magnet. The magnetically attractable shielding particles and the magnet cooperate to provide a shielding system configured to attenuate a radiation dose emitted by the applicator to shield adjacent skin tissue of the patient from the radiation dose.

According to one aspect of the present disclosure, a brachytherapy shielding system includes a brachytherapy applicator having a balloon portion and a catheter portion. The balloon portion may be coupled to the catheter portion and adapted for intracavitary implantation into a patient. Magnetically attractable particles may be configured to be positioned within the balloon portion, and a magnet may be configured to be positioned adjacent to an exterior skin surface of the patient adjacent to the balloon to attract the magnetically attractable particles thereto.

The magnetically attractable particles may be suspended in a liquid. The liquid may include sterile water. Illustratively, the magnetically attractable particles may be one or more of Samarium Cobalt-5 (SmCo₅), Samarium, Gold, Iron, Iron Oxide, and Gadolinium and may be approximately 20-40 nanometers in size. Illustratively, a concentration of the magnetically attractable particles in the liquid may be approximately between 0.1-0.9 g/ml. In some embodiments, the concentration of the magnetically attractable particles in the liquid may be approximately between 0.2-0.7 g/ml. In still other embodiments, the concentration of the magnetically attractable particles in the liquid may be approximately 0.5 g/ml.

According to another aspect of the present disclosure the brachytherapy shielding system includes means for shielding a portion of a patient from radiation emitted from a radiation source within the brachytherapy applicator implanted within the patient to attenuate the amount of radiation received by the portion of the patient. The shielding means may be formed after the brachytherapy applicator is implanted within the patient. At least a portion of the shielding means may be configured to be located within a balloon of the brachytherapy applicator and may be movable relative to the patient after at least a portion of the shielding means is implanted within the patient. The shielding means may include an implanted portion configured to be positioned within a patient and an external portion configured to be positioned outside the patient such that the implanted portion is configured to be spaced apart from the patient. Illustratively, the shielding means may include magnetically attractable particles configured to be positioned within the patient and the magnet configured to be positioned adjacent an external skin surface of the patient to attract the particles thereto.

According to yet another aspect of the present disclosure, a kit for building a brachytherapy shielding system includes magnetically attractable particles for mixing with sterile water to create a liquid mixture, a magnet suitable for medical use for creating a magnetic field to attract the magnetically attractable particles, and instructions for using the magnetically attractable particles and the magnet to create a shield within a brachytherapy applicator in order to reduce the amount of radiation received by a portion of a patient's skin during a brachytherapy treatment. The magnetically attractable particles in the kit may be one or more of Samarium Cobalt-5, Samarium, Gold, Iron, Iron Oxide, and Gadolinium and may be approximately 20-40 nanometers in size.

According to still another aspect of the present disclosure, a method for shielding radiation during radiation treatments using a brachytherapy applicator includes implanting a brachytherapy applicator in a cavity of a patient, and creating a radiation shield within the patient after implanting the brachytherapy applicator. The radiation shield may be created by placing a radiation attenuating material within the brachytherapy applicator. The radiation attenuating material may include magnetically attractable particles creating the radiation shield and creating the radiation shield may further include placing a magnet adjacent an external surface of a patient's skin to attract the magnetically attractable particles thereto.

According to yet another aspect of the present disclosure, a method for shielding radiation during radiation treatments using a brachytherapy applicator includes implanting a brachytherapy applicator in a cavity of a patient, depositing a liquid mixture into a balloon portion of the brachytherapy applicator such that the liquid mixture includes magnetically attractable particles, depositing a radiation source into the balloon portion of the brachytherapy applicator, positioning a magnet adjacent to the outer surface of the skin of the patient, and attracting the magnetically attractable particles to the magnet. This method may further include imaging the patient to determine if the magnetically attractable particles in the brachytherapy applicator are arranged to provide a predetermined amount of radiation shielding, and increasing the concentration of magnetically attractable particles in the liquid mixture in response to a determination that the magnetically attractable particles in the brachytherapy applicator are not arranged to provide the predetermined amount of radiation shielding.

Additional features of the present disclosure will become apparent to those skilled in the art upon consideration of the following detailed description of illustrative embodiments exemplifying the best mode of carrying out the disclosure as presently perceived.

### BRIEF DESCRIPTION OF THE DRAWINGS

The detailed description particularly refers to the accompany figures in which:

FIG. 1 is a diagrammatic view of a brachytherapy shielding system kit in accordance with the present disclosure showing a balloon catheter of a brachytherapy applicator prior to insertion in a lumpectomy cavity, a magnet, and magnetically attractable shielding particles within a liquid mixture;

FIG. 2 is a diagrammatic view of the shielding system of FIG. 1 showing the balloon catheter placed in the lumpectomy cavity and the balloon “inflated” by the liquid mixture; and

FIG. 3 is a view similar to FIG. 2 showing the magnet placed adjacent to a portion of a skin surface of a patient to attract the shielding particles to an interior surface of the balloon within a magnetic field generated by the magnet to shield the skin from radiation effects emitted by radioactive seeds placed in the brachytherapy applicator.

### DETAILED DESCRIPTION

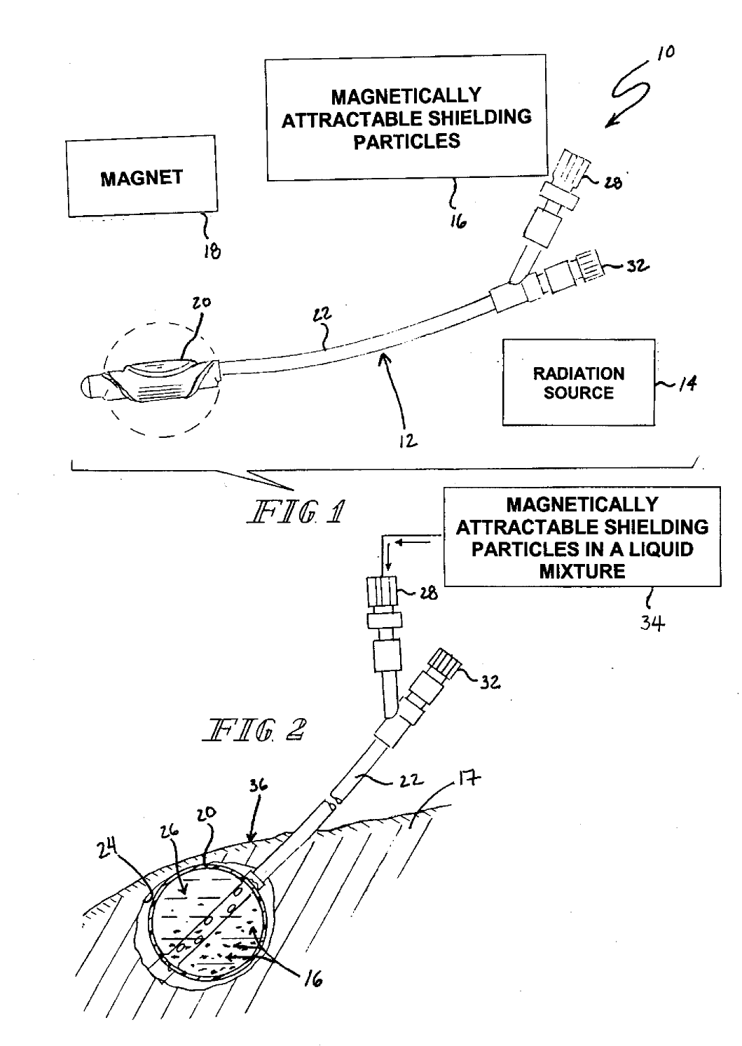

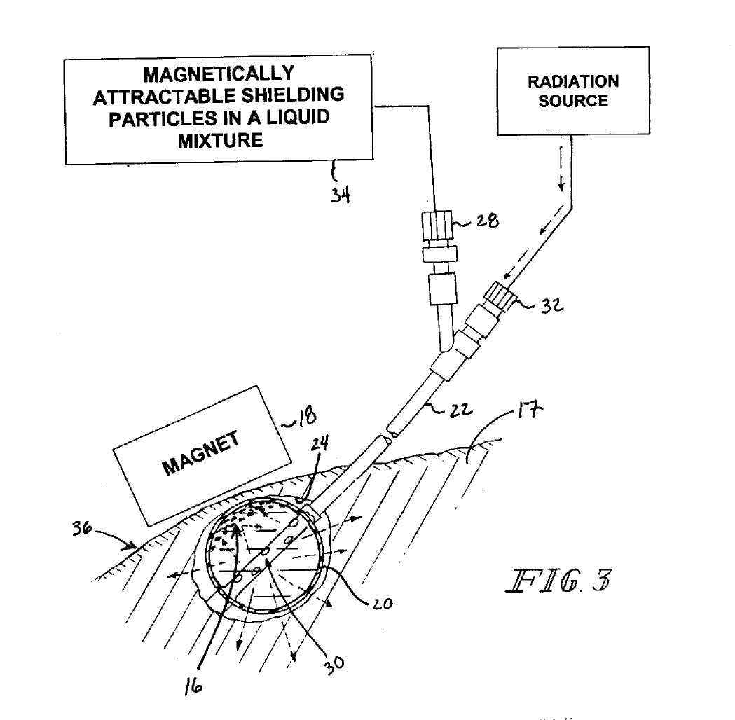

A brachytherapy shielding system 10 includes a brachytherapy applicator 12, a radiation source 14, magnetically attractable shielding particles 16, and a magnet 18 as shown in FIG. 1. The applicator 12 is adapted for intracavital implantation into a patient 17 as shown in FIGS. 2 and 3. The brachytherapy applicator 12 is configured to provide localized radiation therapy to surrounding tissue after a cancerous lump or growth has been removed from the body of the patient such as, for example, in a breast “lumpectomy” or other similar procedure. The shielding system 10 is configured to attenuate a radiation dose emitted by the radiation source 14 to shield skin tissue of the patient 17 (or other portions of the patient's anatomy) adjacent to the brachytherapy applicator 12.

In illustrative embodiments, the applicator 12 is configured as a MammoSite® “balloon catheter” available from Cytyc Corporation of Marlborough, Mass. The applicator 12 includes an inflatable balloon 20 coupled to a catheter 22 as shown in FIG. 1.

The balloon 20 is configured to be placed inside a lumpectomy cavity 24 through a small incision in, for example, the breast of the patient as shown in FIG. 2. A portion of the catheter 22 remains outside the patient, for example, and is secured to a cushioned gauze pad (not shown) to prevent movement of the catheter 22 as shown in FIGS. 2 and 3. The balloon 20 is then “inflated” with a liquid mixture 34 including the magnetically attractable particles 16 which are suspended in a liquid 26, as shown in FIG. 2. through a first catheter port 28 so that it fits snugly into the cavity 24 and generally conforms to the size of the cavity 24. The balloon 20 remains inflated during the duration of the treatment. Initially, the liquid 26 may contain a contrast media so that the balloon 20 is “visible” during computed tomography (CT,) or other suitable types of imaging, of the applicator 12 within the cavity 24 is possible.

In illustrative embodiments, the magnetically attractable particles 16 are one or more of, but not limited to, Samarium Cobalt-5 (SmCo₅), Samarium, Gold, Iron, Iron Oxide, and Gadolinium. In further illustrative embodiments, the magnetically attractable particles 16 are Iron (III) Oxide cosmetic microspheres of about 20-40 nanometers in size. However, any suitable magnetically attractable particle of any suitable size may be used. Illustratively, the liquid 26 is sterile HOH, but may be any suitable liquid. In further illustrative embodiments, a concentration of magnetically attractable particles suspended in liquid is approximately between 0.1-0.9 g/ml. However, other concentrations of approximately 0.2-0.7 g/ml or approximately 0.5 g/ml may be used to provide suitable shielding.

After the liquid mixture 34 is introduced into the balloon, the magnet 18 is placed on or adjacent to an exterior skin surface 36 of the patient 17 adjacent to the balloon 20 within cavity 24. Illustratively, the magnet 18 is placed adjacent to a portion of the patient's skin to be shielded from the radiation emitted by a radiation “seed” 30 placed within the balloon 20 as discussed below. In illustrative embodiments, the magnet 18 is a neodymium iron boron (NdFeB) magnet, however, any magnetic source suitable for medical use may be used. As the magnet 18 is placed on or adjacent to the skin surface 36, the magnetically attractable particles 16 within the liquid mixture 34 are attracted to a magnetic field generated by the magnet 18 and generally agglomerate to form a layer of magnetically attractable particles 16 adjacent an inner surface of the balloon 20, as shown in FIG. 3. Accordingly, the magnetically attractable particles 16 and the magnet 18 cooperate to form a radiation shield after the brachytherapy applicator is implanted within the patient.

Once the layer of magnetically attractable particles 16 is formed, a portion of the catheter 22 that remains outside the breast is coupled to a computer-controlled High Dose Rate (HDR) machine (not shown) that inserts the radiation seed 30 through a second catheter port 32 to deliver the radiation therapy. The radiation seed 30 is deposited through a distal end of catheter 22 into a proximal end of catheter 22 such that the seed 30 is positioned within balloon 20 and thus within the cavity 24. In illustrative embodiments, the radiation seed 30 is Iridium-192, however any suitable radiation source may be used.

As noted above, the magnetically attractable particles 16 are attracted to and agglomerate within the magnetic field of magnet 18 adjacent to an interior surface of balloon 20 between the skin surface 36 and the seed 30. Thus, the magnet 18 and the magnetically attractable particles 16 cooperate to attenuate the radiation dose emitted from the seed 30 as shown in FIG. 3, thereby reducing the amount of radiation received by the portion of the patient's skin or other selected tissue. Illustratively, the position of the magnet 18 adjacent the patient 17 may be moved or adjusted relative to the patient 17 in order to move the layer of magnetically attractable particles 16 and shield a different portion of the patient from the radiation emitted from the seed 30.

Prior to insertion of the radioactive seed 30, the care provider may desire to determine whether a sufficient concentration and agglomeration of magnetically attractable particles 16 is present within balloon 20 to effectively attenuate the radiation emission of seed 30 to shield the adjacent skin or tissue portion of the patient 17 from the radioactive energy. Thus, it may be desirable to subject the patient to CT imaging (or any other suitable imaging) of the brachytherapy applicator 12 while positioned within the lumpectomy cavity 24. If the care provider determines that an insufficient concentration of the magnetically attractable particles 16 is present within the liquid mixture 34, a new concentration of the magnetically attractable particles 16 may be added to the liquid mixture within the balloon 20 to increase the shielding material present.

In some embodiments, the brachytherapy applicator shielding system 10 may include a reflective coating applied to an interior portion of the balloon 20 and capable of reflecting radioactive energy to attenuate the radiation released from the seed 30. The coating may be applied to one or more portions of the balloon corresponding to areas of the patient to be shielded from the radiation. In still other embodiments, a thin flexible magnet 18 may be coupled to or formed on a portion of the balloon 20 for intracavital implantation to attract the magnetically attractable particles 16 within the balloon 20 as selected by the care provider.

Illustratively, a kit for building a brachytherapy shielding system may be provided. Such a kit may include the magnetically attractable particles 16, the magnet 18, and an instruction sheet (not shown) providing instructions for creating a radiation shield. Such instruction sheet may include suggested concentrations for creating the liquid mixture 34 including the particles 16 and sterile water. Such instruction sheet may also include instructions for filling the balloon 20 with the liquid mixture 34, placement and operation of the magnet 18 on the patient, P, and/or use of an imaging maching to determine the thickness of the layer of agglomerated particles 16, for example.

1. A brachytherapy shielding system comprising
a brachytherapy applicator having a balloon portion and a catheter portion, the balloon portion coupled to the catheter portion and adapted for intracavity implantation into a patient,
magnetically attractable particles configured to be positioned within the balloon portion, and
a magnet configured to be positioned adjacent to an exterior skin surface of the patient adjacent to the balloon to attract the magnetically attractable particles thereto.

2. The brachytherapy shielding system of claim 1, wherein the magnetically attractable particles are suspended in a liquid.

3. The brachytherapy shielding system of claim 2, wherein the magnetically attractable particles are one or more of Samarium Cobalt-5, Samarium, Gold, Iron, Iron Oxide, and Gadolinium.

4. The brachytherapy shielding system of claim 2, wherein the magnetically attractable particles are approximately 20-40 nanometers in size.

5. The brachytherapy shielding system of claim 2, wherein the liquid includes sterile water.

6. The brachytherapy shielding system of claim 2, wherein a concentration of the magnetically attractable particles in the liquid is approximately between 0.1-0.9 g/ml.

7. The brachytherapy shielding system of claim 2, wherein a concentration of the magnetically attractable particles in the liquid is approximately between 0.2-0.7 g/ml.

8. The brachytherapy shielding system of claim 2, wherein a concentration of the magnetically attractable particles in the liquid is approximately 0.5 g/ml.

9. A brachytherapy shielding system comprising
means for shielding a portion of a patient from radiation emitted from a radiation source located within a brachytherapy applicator implanted within the patient to attenuate the amount of radiation received by the portion of the patient, the shielding means being formed after the brachytherapy applicator is implanted within the patient.

10. The brachytherapy shielding system of claim 9, wherein at least a portion of the shielding means is configured to be located within a balloon of the brachytherapy applicator.

11. The brachytherapy shielding system of claim 9, wherein the shielding means is movable relative to the patient after at least a portion of the shielding means is implanted within the patient.

12. The brachytherapy shielding system of claim 9, wherein the shielding means includes an implanted portion configured to be positioned within a patient and an external portion configured to be positioned outside the patient.

13. The brachytherapy shielding system of claim 12, wherein the implanted portion is configured to be spaced apart from the patient.

14. The brachytherapy shielding system of claim 9, wherein the shielding means includes magnetically attractable particles configured to be positioned within the patient and a magnet configured to be positioned adjacent an external skin surface of the patient to attract the particles thereto.

15. The brachytherapy shielding system of claim 14, wherein the magnetically attractable particles are one or more of Samarium Cobalt-5, Samarium, Gold, Iron, Iron Oxide, and Gadolinium approximately 20-40 nanometers in size.

16. A kit for building a brachytherapy shielding system comprising
magnetically attractable particles for mixing with sterile water to create a liquid mixture, wherein the magnetically attractable particles are approximately 20-40 nanometers
a magnet suitable for medical use for creating a magnetic field to attract the magnetically attractable particles, and
instructions for using the magnetically attractable particles and the magnet to create a shield within a brachytherapy applicator in order to reduce the amount of radiation received by a portion of a patient's skin during a brachytherapy treatment.

17. The kit of claim 16 wherein the magnetically attractable particles are one or more of Samarium Cobalt-5, Samarium, Gold, Iron, Iron Oxide, and Gadolinium.

18. A method for shielding radiation during radiation treatments using a brachytherapy applicator comprising the steps of
implanting a brachytherapy applicator in a cavity of a patient,
creating a radiation shield within the patient after implanting the brachytherapy applicator.

19. The method of claim 18, wherein creating the radiation shield includes placing a radiation attenuating material within the brachytherapy applicator.

20. The method of claim 19, wherein the radiation attenuating material includes magnetically attractable particles creating the radiation shield and creating the radiation shield further includes placing a magnet adjacent an external surface of a patient's skin to attract the magnetically attractable particles thereto.

21. A method for shielding radiation during radiation treatments using a brachytherapy applicator comprising the steps of
implanting a brachytherapy applicator in a cavity of a patient,
depositing a liquid mixture into a balloon portion of the brachytherapy applicator, wherein the liquid mixture includes magnetically attractable particles,
depositing a radiation source into the balloon portion of the brachytherapy applicator,
positioning a magnet adjacent to the outer surface of the skin of the patient, and
attracting the magnetically attractable particles to the magnet.

22. The method of claim 21, further comprising the steps of
imaging the patient to determine if the magnetically attractable particles in the brachytherapy applicator are arranged to provide a predetermined amount of radiation shielding, and
increasing the concentration of magnetically attractable particles in the liquid mixture in response to a determination that the magnetically attractable particles in the brachytherapy applicator are not arranged to provide the predetermined amount of radiation shielding.
